(19) United States
(12) Patent Application Publication
(10) Pub. No.: US 2024/0334816 A1
(43) Pub. Date: Oct. 3, 2024

(54) HOLE TRANSPORT MATERIAL, AND PHOTOELECTRIC CONVERSION ELEMENT AND ORGANIC SOLAR CELL USING HOLE TRANSPORT MATERIAL

(71) Applicant: KYOCERA Document Solutions Inc., Osaka (JP)

(72) Inventors: Jun AZUMA, Osaka (JP); Kenji KITAGUCHI, Osaka (JP)

(73) Assignee: KYOCERA Document Solutions Inc., Osaka (JP)

(21) Appl. No.: 18/623,705

(22) Filed: Apr. 1, 2024

(30) Foreign Application Priority Data
Apr. 3, 2023 (JP) 2023-060001

(51) Int. Cl. H10K 85/60; H10K 30/40; H10K 30/86

(52) U.S. Cl. CPC H10K 85/631; H10K 30/40; H10K 30/86

(57) ABSTRACT

Provided is a hole transport material represented by a general formula (1) below. In the general formula (1), R₁ represents an alkoxy group or an alkyl group with four or fewer carbon atoms, R₂ represents a hydrogen atom or a methyl group, and a total number of carbon atoms in R₁ and R₂ is two or more.

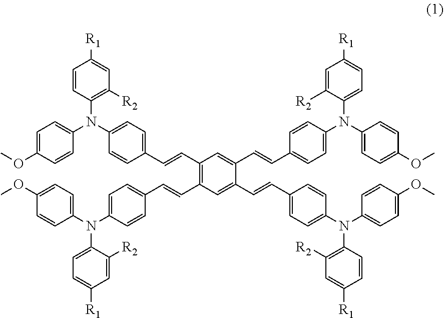

### INCORPORATION BY REFERENCE

This application is based on and claims the benefit of priority from Japanese Patent Application No. 2023-060001 filed on Apr. 3, 2023, the contents of which are hereby incorporated by reference.

### BACKGROUND

The present disclosure relates to a hole transport material used as a material for hole transport layers, and a photoelectric conversion element and an organic solar cell using the hole transport material.

In recent years, the drive power required by electronic circuits has become very small, so that various electronic components such as sensors can be driven even with a weak electric power. Furthermore, in utilization of sensors, sensors are expected to be applied to self-supporting power supplies that can generate power on the spot for its own power consumption, and among such self-supporting power supplies, solar cells have attracted attention as elements capable of generating power anywhere as long as light is available.

As solar cells, inorganic solar cells and organic solar cells are known. However, inorganic solar cells, which utilize inorganic semiconductor materials such as silicon for both p-type and n-type semiconductors, unfortunately have their usage constrained due to the high production cost and the difficulty in scaling up. Consequently, advancements are being made today in the development of organic solar cells, which utilize organic semiconductors instead of inorganic ones. Organic solar cells, for example, can be classified into categories such as dye-sensitized solar cells, organic thin-film solar cells, and organic-inorganic hybrid solar cells.

Organic solar cells contain p-type and n-type semiconductors, and are often provided with a hole transport layer formed between a photoelectric conversion layer, which absorbs light to generate positive and negative charges, and a positive terminal. The hole transport layer facilitates efficient movement of photoexcited charges, positive (holes) and negative (electrons), preventing their recombination, and in this manner, the hole transport layer plays a role in improving the photoelectric conversion efficiency of solar cells.

Among organic solar cells, in particular, those (hereinafter referred to also as perovskite-type solar cells) provided with a photoelectric conversion layer containing a perovskite compound have been reported to exhibit photoelectric conversion efficiency that is equal to or higher than the photoelectric conversion efficiency of amorphous-silicon solar cells in weak indoor-light environments, and also, successive reports have been made on further improvements in photoelectric conversion efficiency of perovskite-type solar cells. The basic structure of perovskite-type solar cells generally is a multilayered body structure where a transparent electrode (a negative terminal), an electron transport layer, a photoelectric conversion layer (a perovskite layer), a hole transport layer, and a metal electrode (a positive terminal) are stacked in this order. This structure may be provided with a mesoporous titania layer between the electron transport layer and the perovskite layer such that the perovskite layer and the mesoporous titania layer together constitute a photoelectric conversion layer. Among these layers, the hole transport layer is generally constituted of a material that contains an organic semiconductor.

As organic semiconductors to be contained in the materials of hole transport layers that have been reported so far, for example, 2,2′,7,7′-tetrakis-(N,N-di-methoxyphenylamine)-9,9′-spirobifluorene (hereinafter also referred to as Spiro-OMeTAD) is known which has been developed as a material for hole transport layers for dye-sensitized solar cells, and it is often used also in perovskite-type solar cells mentioned previously.

### SUMMARY

According to one aspect of the present disclosure, a hole transport material is represented by a general formula (1) below:

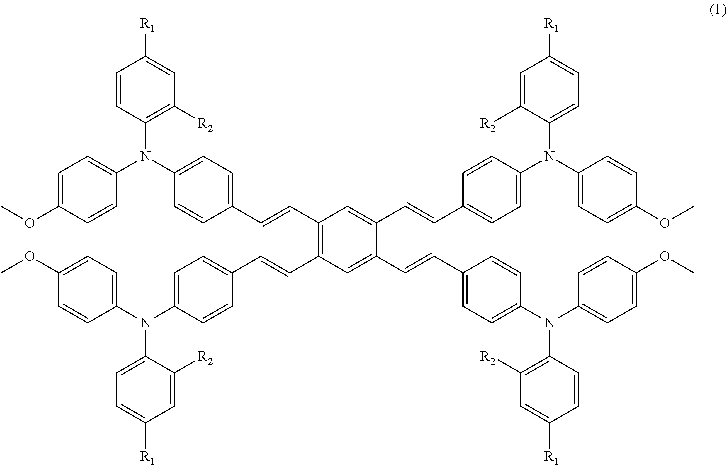

In the general formula (1), R₁ represents an alkoxy group or an alkyl group with four or fewer carbon atoms, R₂ represents a hydrogen atom or a methyl group, and a total number of carbon atoms in R₁ and R₂ is two or more.

### BRIEF DESCRIPTION OF THE DRAWINGS

FIG. 1 is a partial sectional view of an organic solar cell according to an embodiment of the present disclosure, illustrating an example where a first electrode is an electron injection electrode, and a second electrode is a hole injection electrode;

FIG. 2 is a partial sectional view of the organic solar cell according to the embodiment of the present disclosure, illustrating an example where the first electrode is the hole injection electrode, and the second electrode is the electron injection electrode;

FIG. 3 is a schematic diagram illustrating a measurement device for measuring the hole mobility of a hole transport layer by the MIS-CELIV method;

FIG. 4 is an explanatory diagram illustrating results of the hole mobility measurement by the MIS-CELIV method, a graph illustrating time-series change of a reverse voltage that increases linearly;

FIG. 5 is an explanatory diagram illustrating results of the hole mobility measurement by the MIS-CELIV method, a graph illustrating time-series change of a transient current generated as a result of application of the reverse voltage;

FIG. 6 is a graph illustrating waveforms of transmission currents observed in a case where the reverse voltage application speed A=150 kV/s and a forward voltage V_{FB}=0 V to −10V; and

FIG. 7 is a graph illustrating electric field dependence of the hole mobility estimated from waveforms of transient currents observed under conditions where the reverse voltage application speed A=100 to 350 kV/s, and the forward voltage V_{FB}=−10 V.

### DETAILED DESCRIPTION

### [1. Configuration of Organic Solar Cell]

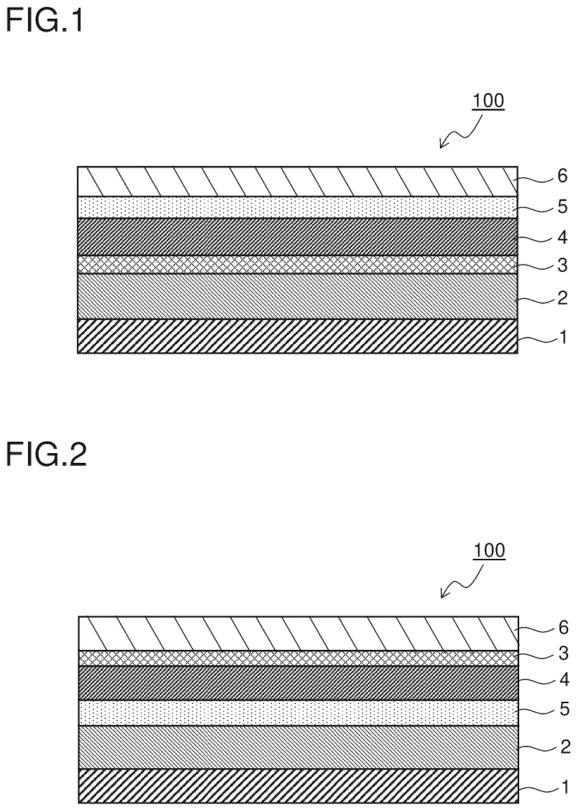

First, a solar cell will be described in which a hole transport material of the present disclosure is used. FIG. 1 is a partial sectional view of an organic solar cell 100 according to an embodiment of the present disclosure. The organic solar cell 100 (hereinafter simply referred to as the solar cell 100) includes a substrate 1, a first electrode 2, an electron transport layer 3, a photoelectric conversion layer 4, a hole transport layer 5, and a second electrode 6. The first electrode 2 is provided on the substrate 1. The photoelectric conversion layer 4 is stacked over the first electrode 2 via the electron transport layer 3. The second electrode 6 is stacked over the photoelectric conversion layer 4 via the hole transport layer 5. Note that, without providing an electron transport layer, the photoelectric conversion layer 4 may be stacked directly on the first electrode 2. In that case, the electron transport layer 3 illustrated in FIG. 1 is omitted. When the solar cell 100 is in use, for example, a surface of the solar cell 100 on the substrate 1 side is irradiated with light L (e.g., solar light or indoor light). Note that, when the solar cell 100 is in use, a surface of the solar cell 100 on the second electrode 6 side may be irradiated with the light L.

### <Substrate>

There is no particular limitation on the substrate 1, as long as it is usable in the solar cell 100. The substrate 1 may be transparent or may be opaque. However, in the case where the surface of the solar cell 100 on the substrate 1 side is the light receiving surface, it is preferable that the substrate 1 be transparent. Examples of transparent substrates include transparent rigid substrates including glass such as quartz glass, synthetic quartz plates, etc., and transparent flexible substrates including transparent resin films, optical resin plates, etc. Transparent flexible substrates are advantageous in that they are easy to process, they contribute to lower production cost and lighter weight, they are difficult to be broken, and they are applicable to curved surfaces.

### <First Electrode>

The first electrode 2 is an electron injection electrode, for example. The first electrode 2 is not limited to a conductive material. Examples of materials used as the first electrode 2 which are high in work function include gold (Au), silver (Ag), cobalt (Co), nickel (Ni), platinum (Pt), carbon (C), indium tin oxide (ITO), tin oxide (SnO₂), fluorine-doped tin oxide (FTO), and zinc oxide (ZnO).

Further, the material for the first electrode 2 is appropriately chosen by considering whether the light receiving surface of the solar cell 100 is the surface on the substrate 1 side or the surface on the second electrode 6 side. In a case where the light receiving surface is the surface on the substrate 1 side, it is preferable that the first electrode 2 be a transparent electrode. Examples of materials used when the first electrode 2 is a transparent electrode include indium zinc oxide (IZO), ITO, FTO, ZnO—Al, and Zn—Sn—O.

The total light transmittance of the first electrode 2 is preferably 85% or higher, more preferably 90% or higher, and particularly preferably 92% or higher. When the total light transmittance of the first electrode 2 is 85% or higher, if the light receiving surface of the solar cell 100 is the surface on the substrate 1 side, light can sufficiently pass through the first electrode 2 on the substrate 1 to be efficiently absorbed by the photoelectric conversion layer 4.

Sheet resistance of the first electrode 2 is preferably 20 Ω/sq or lower, and more preferably 15 Ω/sq or lower. When the sheet resistance of the first electrode 2 is 20 Ω/sq or lower, charge generated in the photoelectric conversion layer 4 is sufficiently transferred to an external circuit. The sheet resistance can be measured, for example, using a resistivity meter (Loresta AXMCP-T370, manufactured by Nittoseiko Analytech Co., Ltd., 4-probe type), by a method compliant with JIS (Japanese Industrial Standards) R1637 (method of resistivity test for fine ceramics thin film-measurement method by 4-probe method).

Film thickness of the first electrode 2 is preferably 0.1 nm or more but 500 nm or less, and more preferably 1 nm or more but 300 nm or less. When the film thickness of the first electrode 2 is 0.1 nm or more, the sheet resistance of the first electrode 2 does not become too high, and the charge (holes) generated in the photoelectric conversion layer 4 can be sufficiently transferred to the external circuit. On the other hand, when the film thickness of the first electrode 2 is 500 nm or less, total light transmittance of the first electrode 2 becomes high.

The first electrode 2 may be single-layered. Or, the first electrode 2 may be composed of a plurality of layers having different work functions. In the case where the first electrode 2 is composed of a plurality of layers, the term “film thickness of the first electrode 2” mentioned above refers to a total thickness of the plurality of layers.

The first electrode 2 may be formed in a sheet-shape over the whole area of the substrate 1, or may be formed in a pattern shape on the substrate 1. Or, the first electrode 2 may be flat-shaped, or may be uneven-shaped. Examples of the uneven shape include texture-structure shapes, pyramid-structure shapes, wave-structure shapes, comb-structure shapes, and nanopillar-structure shapes. In a case where the first electrode 2 has an uneven shape, incident light is scattered by the unevenness of the surface of the first electrode 2, and thus an increased amount of light is taken into the photoelectric conversion layer 4, which helps improve energy conversion efficiency of the solar cell 100.

### <Electron Transport Layer>

The electron transport layer 3 is stacked between the first electrode 2 and the photoelectric conversion layer 4. The electron transport layer 3 is provided so as to facilitate the injection (movement) of electrons from the photoelectric conversion layer 4 into the first electrode 2 (an electron injection electrode). The provision of the electron transport layer 3 helps enhance the efficiency with which electrons are injected from the photoelectric conversion layer 4 into the first electrode 2, and thus the energy conversion efficiency of the solar cell 100 is improved.

There is no particular limitation on a material (an electron transport layer material) to be contained in the electron transport layer 3, as long as the material allows stable injection of electrons from the photoelectric conversion layer 4 into the first electrode 2. Examples of the electron transport layer material include conductive organic compounds, charge transfer complexes, alkali metals, alkaline earth metals, and organic compounds doped with an alkali metal or an alkaline earth metal, and metal oxides.

Examples of alkali metals usable as an electron transport layer material include lithium, sodium, potassium, rubidium, and cesium. Examples of alkaline earth metals usable as an electron transport layer material include beryllium, magnesium, calcium, strontium, and barium. Examples of organic compounds doped with an alkali metal or an alkaline earth metal usable as an electron transport layer material include bathocuproine (BCP) and bathophenanthroline (Bphen) doped with an alkali metal or an alkaline earth metal. The alkali metal or the alkaline earth metal used for doping is preferably lithium, sodium, potassium, rubidium, cesium, beryllium, magnesium, calcium, strontium, or barium, and more preferably, lithium, cesium, barium, or strontium. Examples of metal oxides usable as an electron transport material include titanium oxide, zinc oxide, tin oxide, etc.

In a case where the electron transport layer material is a conductive organic compound, a charge transfer complex, or a metal oxide, the electron transport layer 3 preferably has a film thickness that is 10 nm or more but 200 nm or less. In a case where the electron transport layer material is an alkali metal, an alkaline earth metal, or an organic compound doped with an alkali metal or an alkaline earth metal, the electron transport layer 3 preferably has a film thickness that is 0.1 nm or more but 50 nm or less.

### <Photoelectric Conversion Layer>

The photoelectric conversion layer 4 may be a bulk heterojunction type photoelectric conversion layer containing a donor (an electron donor) and an acceptor (an electron acceptor), or may be what is called a perovskite, which is a photoelectric conversion layer having a perovskite layer.

In a case of a bulk heterojunction type photoelectric conversion layer, although there is no particular limitation on the donor as long as it functions as a donor, the donor is preferably a conductive polymer (an electron-donating organic material) having an electron-donating characteristic. An electron-donating organic material is one of two organic compounds that has lower electron affinity than the other when they are used in contact with each other. That is, any organic compound is usable as the donor as long as it has an electron donating characteristic. The donor is preferably a compound which can be formed into a film by a method (e.g., a coating method such as a casting method and a spin coating method) of forming a thin film using a solution obtained by dissolving a donor in an organic solvent. Note that the photoelectric conversion layer 4 may contain only a donor and an acceptor, or may further include another compound.

Examples of electron-donating conductive polymers usable as the donor include polyphenylene, polyphenylene vinylene, polysilane, polythiophene, polybenzodithiophene, polycarbazole, polyvinylcarbazole, porphyrin, polyacetylene, polypyrrole, polyaniline, polyfluorene, polyvinylpyrene, polyvinylanthracene, and their derivatives. The donor may be a copolymer formed by copolymerization of at least two of these electron-donating conductive polymers. Further, other examples of electron-donating conductive polymers include a phthalocyanine-containing polymer, a carbazole-containing polymer, and an organometallic polymer.

Preferable as an electron-donating conductive polymer usable as the donor is a polymer (a polythiophene-based polymer) that has at least one of a thiophene structure, a benzothiophene structure, and a benzodithiophene structure. The polythiophene-based polymer is preferably capable of absorbing visible light. Further, the polythiophene-based polymer is preferably of a donor-acceptor (DA) type.

Although there is no particular limitation on the acceptor as long as it functions as an acceptor, the acceptor is preferably an electron-accepting conductive polymer (an electron-accepting organic material). Electron-accepting organic materials, of which typical representatives are electron-transporting organic compounds, refer to organic compounds that readily accept electrons. More specifically, an electron-accepting organic material is one of two organic compounds that has higher electron affinity than the other when they are used in contact with each other. That is, any organic compound is usable as the acceptor as long as it is an electron-accepting organic compound.

Examples of preferably used electron-accepting organic materials include fullerene and a derivative thereof (such as PCBM), carbon nanotube and a derivative thereof, perylene and a derivative thereof (such as PTCDA, PTCDI), naphthalene derivative (such as NTCDA, NTCDI or the like), oligomers or polymers having a backbone that includes pyridine or derivatives thereof, fluorinated metal-free phthalocyanine, fluorinated metal phthalocyanines and derivatives thereof, tris (8-hydroxyquinolinato) aluminum complexes, bis (4-methyl-8-quinolinato) aluminum complexes, distyrylarylene derivatives, silole compounds, etc., among which, in particular, fullerene-based derivatives (such as PCBM) are preferably, but not limitedly, used.

In the photoelectric conversion layer 4, a ratio (MA/MD) of the mass of the acceptor (MA) with respect to the mass of the donor (MD) is preferably 0.1 or higher but 2.0 or lower, and more preferably 1.0 or higher but 2.0 or lower. With the ratio (MA/MD) within this range, the acceptor and the donor of the solar cell 100 are well-balanced, and thus the energy conversion efficiency of the solar cell 100 is improved.

A photoelectric conversion layer having a perovskite layer uses a compound represented by a general formula (ABX₃). This compound may be, for example, a perovskite crystal or a perovskite complex. A represents a monovalent cation formed from an organic amino compound such as methylamine, ethylamine, n-butylamine, di-n-butylamine, trimethylamine, triethylamine, methyl-n-hexylamine, methyldiethylamine, tri-n-hexylamine, imidazole, pyrrole, aziridine, formamidine, guanidine, pyridine, 4-t-butylpyridine, phenethylamine, 5-aminovaleric acid, or the like, or a monovalent cation formed from cesium, potassium, rubidium, or the like, which may be used alone or as a mixture of two or more. B represents a divalent cation of lead, tin, or the like, which may be used alone or as a mixture. Further, it may be mixed with a small amount of trivalent cation or indium, antimony, or the like. X represents an atom of halogen such as chlorine, bromine, iodine, or the like, or an anion source, which may be used alone or as a mixture of two or more.

On the film thickness of the photoelectric conversion layer 4, whether it is a bulk heterojunction type or a perovskite type, there is no particular limitation as long as a desired energy conversion efficiency can be achieved. The film thickness of the photoelectric conversion layer 4 is preferably 0.2 nm or more but 3000 nm or less, and more preferably 10 nm or more but 600 nm or less. When the photoelectric conversion layer 4 has a film thickness that is 3000 nm or less, the sheet resistance of the photoelectric conversion layer 4 is likely to have a desired value. On the other hand, when the photoelectric conversion layer 4 has a film thickness that is 0.2 nm or more, the first electrode 2 and the second electrode 6 are unlikely to be short-circuited.

### <Hole Transport Layer>

The hole transport layer 5 is stacked between the photoelectric conversion layer 4 and the second electrode 6. The hole transport layer 5 is provided so as to facilitate the injection (movement) of holes from the photoelectric conversion layer 4 into the second electrode 6 (a hole injection electrode). The provision of the hole transport layer 5 helps enhance the efficiency with which holes are injected from the photoelectric conversion layer 4 into the second electrode 6, as a result of which the energy conversion efficiency of the solar cell 100 is improved. The hole transport layer 5 contains a 1, 2, 4, 5-tetrakis (6-diphenylamino) styrylbenzene derivative represented by a general formula (1) below. The hole transport layer 5 may contain only a 1,2,4,5-tetrakis (6-diphenylamino) styrylbenzene derivative represented by the general formula (1), or may further include an additive. As the additive, a conventionally known additive can be used such as a surface treatment agent.

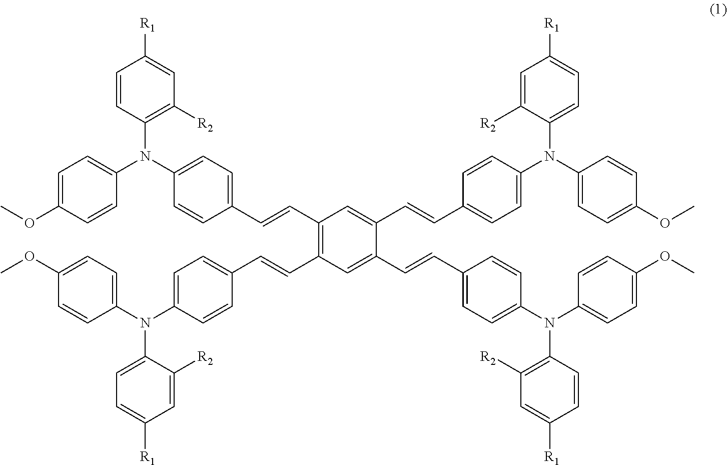

In the general formula (1), R₁ represents an alkoxy group or an alkyl group with four or fewer carbon atoms, R₂ represents a hydrogen atom or a methyl group, and a total number of carbon atoms in R₁ and R₂ is two or more.

Specific examples of 1, 2, 4, 5-tetrakis (6-diphenylamino) styrylbenzene derivatives represented by the general formula (1) include compounds HTM-1 to HTM-7 represented by the following formulae.

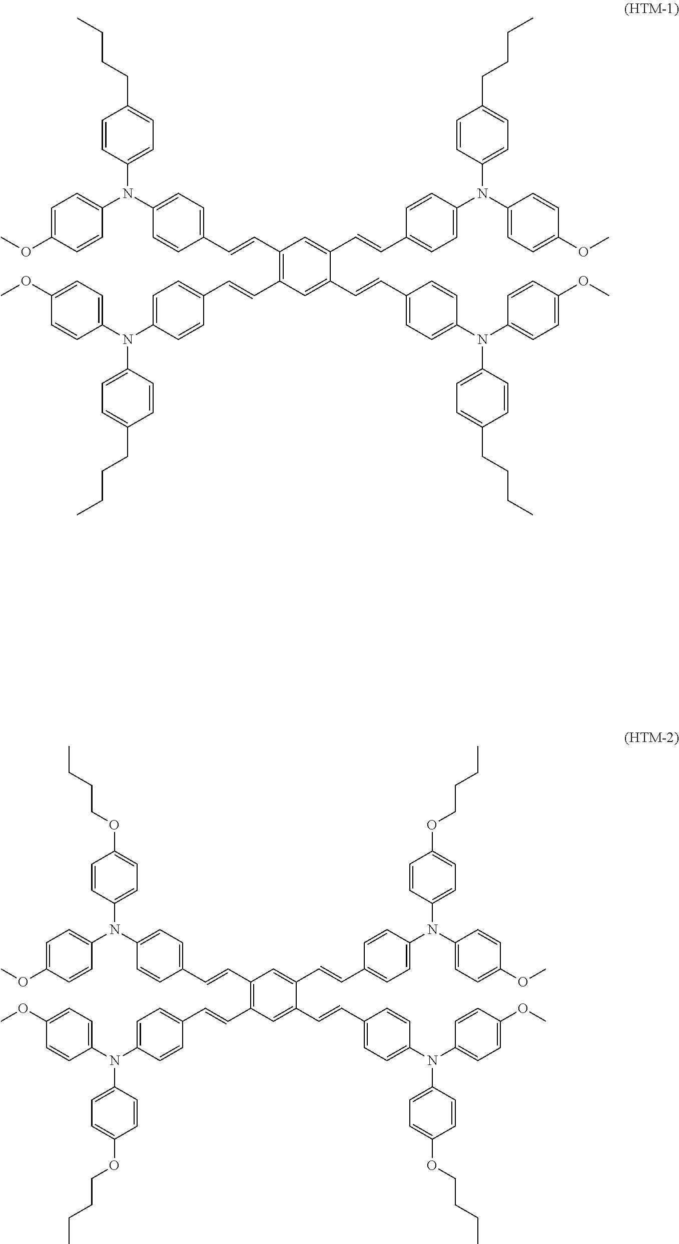

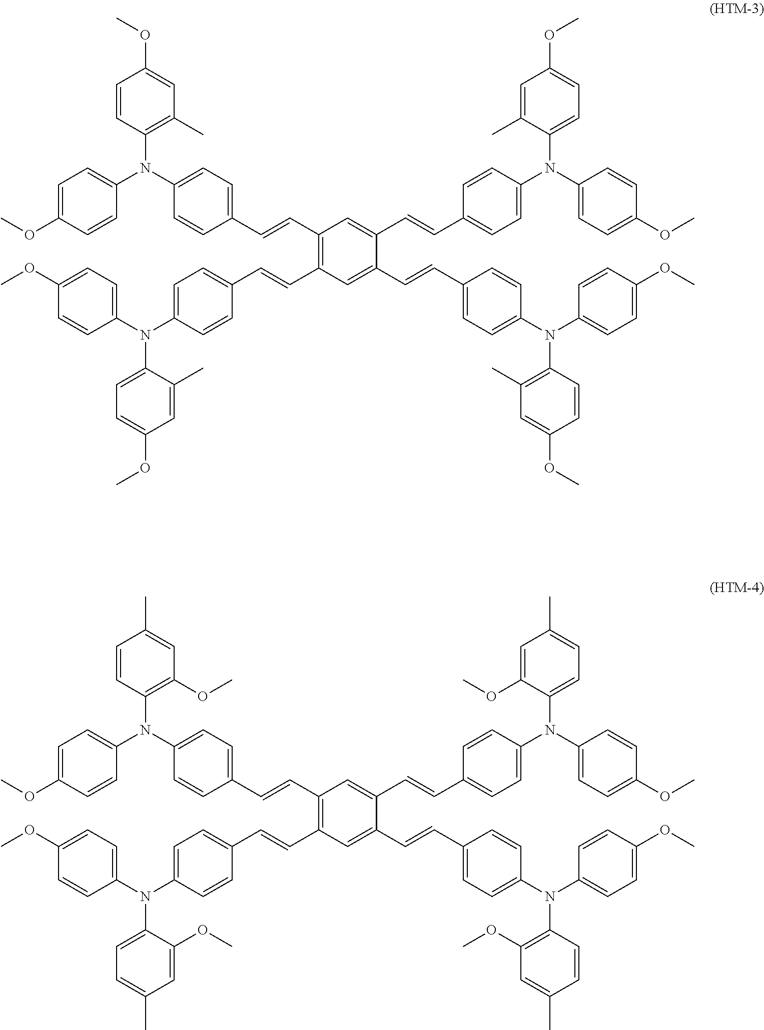

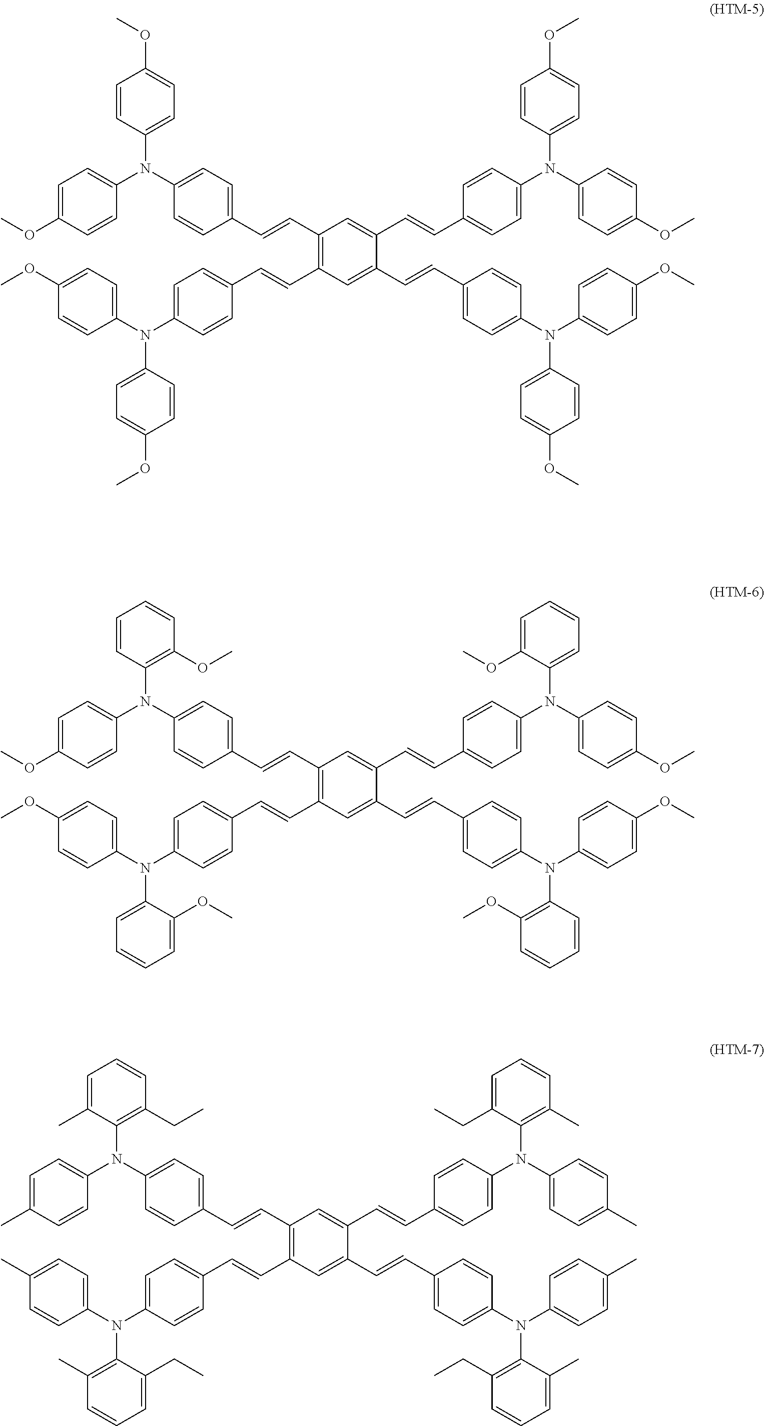

Using a compound represented by the general formula (1) above as the hole transport member constituting the hole transport layer 5, it is possible to improve the mobility of charge (holes), as a result of which the energy conversion efficiency of the solar cell 100 is improved. As to the mobility of charge (holes), a description will be given later.

### <Second Electrode>

The second electrode 6 is a hole injection electrode, for example. The second electrode 6 is provided opposite the first electrode 2. There is no particular limitation on the second electrode 6, as long as it has conductivity. A material for the second electrode 6 is appropriately selected by considering, for example, a work function of the hole transport layer 5. In a case where the material for the hole transport layer 5 is a material having a high work function, the material for the second electrode 6 is preferably a material having a low work function.

Examples of materials to be used as the material for the second electrode 6 include gold (Au), silver (Ag), cobalt (Co), nickel (Ni), platinum (Pt), carbon (C), indium tin oxide (ITO), tin oxide (SnO₂), fluorine-doped tin oxide (FTO), and zinc oxide (ZnO).

Further, the material for the second electrode 6 is appropriately selected by considering, for example, whether the light receiving surface of the solar cell 100 is the surface on the substrate 1 side or the surface on the second electrode 6 side. In the case where the light receiving surface is the surface on the substrate 1 side, the first electrode 2 is preferably a transparent electrode, but the second electrode 6 does not necessarily need to be a transparent electrode.

The film thickness of the second electrode 6 is preferably 0.1 nm or more but 500 nm or less, and more preferably 1 nm or more but 300 nm or less. If the second electrode 6 has a film thickness that is 0.1 nm or more, the sheet resistance of the second electrode 6 does not become too high, and thus the charge generated in the photoelectric conversion layer 4 can be sufficiently transferred to an external circuit.

The second electrode 6 may be single-layered. Or, the second electrode 6 may be composed of a plurality of layers having different work functions. In a case where the second electrode 6 is composed of a plurality of layers, the term “film thickness of the second electrode 6” mentioned above refers to a total thickness of the plurality of layers.

The second electrode 6 may be formed in a sheet-shape over the whole area of the hole transport layer 5, or may be formed in a pattern shape on the hole transport layer 5.

### <Other Components>

The solar cell 100, as necessary, may also include other components in addition to the substrate 1, the first electrode 2, the electron transport layer 3, the photoelectric conversion layer 4, the hole transport layer 5, and the second electrode 6, all of which have been described above. Examples of other components include a protection sheet layer, a filler material layer, a barrier layer, a protection hard coat layer, a strength support layer, an antifouling layer, a high reflectance layer, a light containment layer, an ultraviolet blocking layer, an infrared blocking layer, and a sealing material layer. Further, in the solar cell 100, adhesion layers may be stacked between layers as necessary.

Further, the solar cell 100 is not limited to the structure shown in FIG. 1, but instead, for example, the first electrode 2 may be a hole injection electrode, and the second electrode 6 may be an electron injection electrode. In that case, the hole transport layer 5 and the electron transport layer 3 are stacked in the order opposite to the order in FIG. 1. Specifically, as shown in FIG. 2, the hole transport layer 5 is stacked between the first electrode 2 and the photoelectric conversion layer 4, and the electron transport layer 3 is stacked between the photoelectric conversion layer 4 and the second electrode 6.

### [2. Measurement of Hole Mobility of Hole Transport Material]

Next, a description will be given of the mobility of charge (holes) of the hole transport material constituting the hole transport layer 5. The mobility refers to the speed of carrier dispersion, and a higher mobility of the hole transport material improves the distance by which carriers can disperse, seemingly giving a higher photoelectric conversion efficiency in perovskite-type solar cells, for example. Methods for measuring the mobility include a time of flight (TOF) method, a field-effect transistor (FET) method in which field effect transistors are fabricated and evaluated, a photo-CELIV method, and a MIS-CELIV method as described in AIP ADVANCES 8, 105001 (2018).

These methods each have its own advantages and disadvantages. For example, the time of flight method requires forming a thick film, about 1 μm in thickness, and thus, in this method, it is difficult to use a coating method to form a film. For comparatively accurate and easy measurement with a coating film having a thickness of 1 μm or less, evaluation is preferably performed by a photo-CELIV method or the MIS-CELIV (injection-charge extraction by linearly increasing voltage in metal-insulator-semiconductor structures) method.

The MIS-CELIV method is a technique that includes accumulating externally injected charge at an insulator layer interface and estimating the mobility of electrons or holes from a transient-current waveform which results from extraction of the accumulated charge, and this method is anticipated to be a new measurement technique that enables the analysis of organic semiconductor thin films. Hereinafter, a description will be given of a hole mobility evaluation method using the MIS-CELIV method.

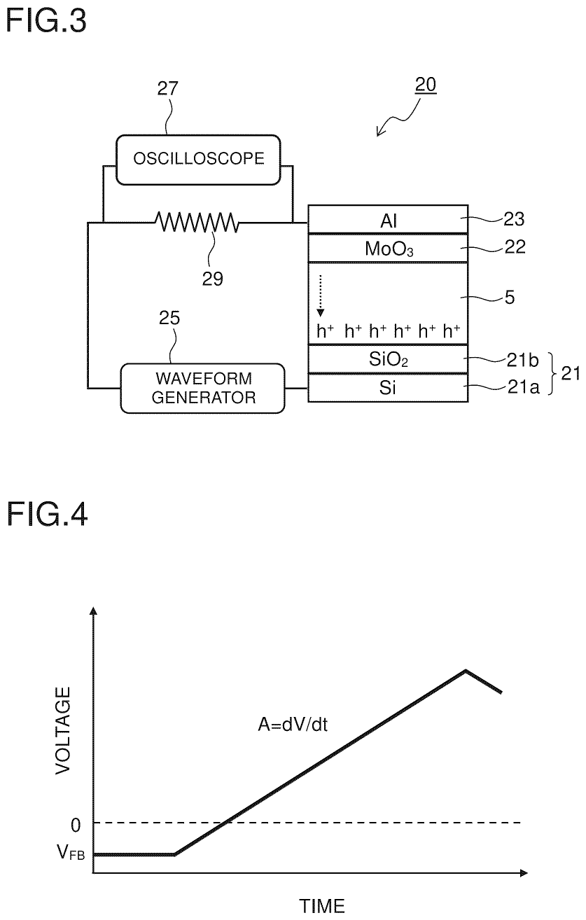

FIG. 3 is a schematic diagram illustrating a measurement device 20 for measuring the hole mobility of a hole transport layer by the MIS-CELIV method. As a measurement substrate 21, a highly n-doped silicon wafer having a thermal oxide silicon (SiO₂) layer 21b stacked on a surface of an Si layer 21a. On a surface of this measurement substrate 21, the hole transport layer 5 was formed by a spin coat method. Subsequently, on a surface of the hole transport layer 5, a hole injection layer 22 of molybdenum trioxide (MoO₃) and an Al electrode 23 were sequentially deposited. Then, to the Si layer 21a and the Al electrode 23, a circuit was connected which included a waveform generator 25, an oscilloscope 27, and a resistor 29. Film thicknesses of the layers were as follows: the SiO₂ layer 21b, 30 nm; the hole transport layer 5, approximately 100 nm; the hole injection layer 22 (MoO₃), 5 nm; and the Al electrode 23, 100 nm. These film thicknesses were measured with a stylus profiler (Dektak XT, manufactured by Bruker Corporation).

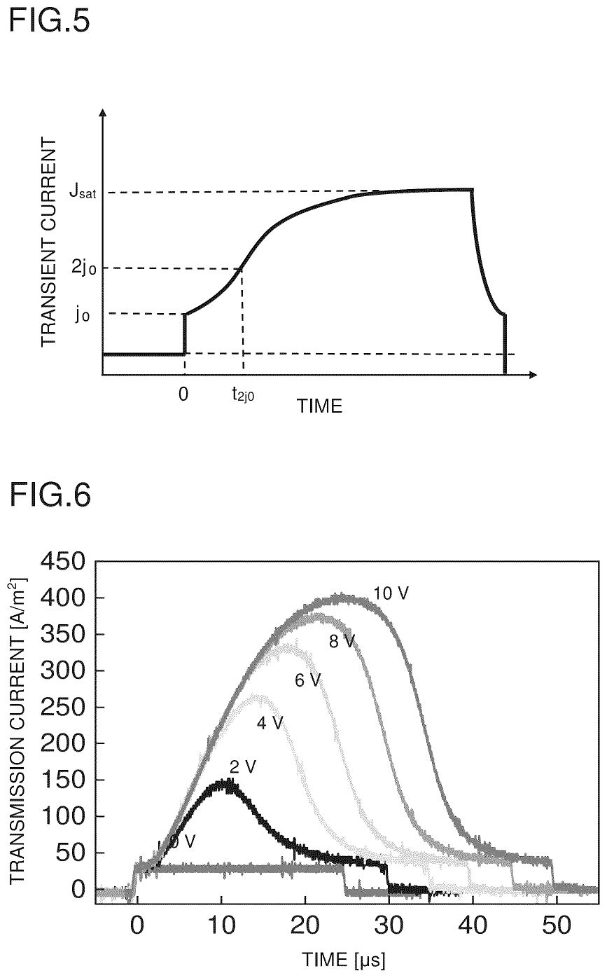

FIG. 4 and FIG. 5 are explanatory diagrams illustrating results of hole-mobility measurement by the MIS-CELIV method, graphs respectively illustrating time-series change of the reverse voltage and time-series change of the transient currents. When a negative forward voltage V_{FB} is applied to the measurement substrate 21, holes h⁺ are injected from the Al electrode 23, via the hole injection layer 22, into the hole transport layer 5, and, as shown in FIG. 3, the holes h⁺ are accumulated at the interface between the SiO₂ layer (an insulator layer) 21b and the hole transport layer (a semiconductor) 5.

In this state, by applying a reverse voltage that linearly increases at a voltage rise speed A=dV/dt as shown in FIG. 4, the accumulated holes h⁺ can be extracted. Transient phenomena of the MIS-CELIV method consist of a displacement current j₀, which is due to a total geometric capacitance of an insulator and a semiconductor, and a current peak Δj by a saturation current jₛₐₜ, which is due to extraction of the accumulated holes h⁺. That is, by the saturation current jₛₐₜ due to the extraction of the accumulated holes h⁺, the current peak Δj is determined. Hole mobility μ is determined by a formula (2) below.

$$μ = \frac{2 {d}_{s}^{2}}{{At}_{tr}^{2}} ( 1 + \frac{{ε}_{s} {d}_{i}}{{ε}_{i} {d}_{s}} ) \qquad ( 2 )$$

In the formula (2), εₛ represents a dielectric constant of the semiconductor, εᵢ represents a dielectric constant of the insulator layer, dₛ represents a film thickness of the semiconductor, dᵢ represents a film thickness of the insulator layer, and A represents a reverse voltage application speed. A carrier transport time tₜᵣ is related to a characteristic time t₂ⱼ₀ until the value of j₀ doubles.

In a case where a capacitance Ci of the insulator layer is sufficiently larger than a capacitance Cs of the semiconductor (Ci/Cs≥1), tₜᵣ is defined by a formula (3) below, with consideration given to applied voltage reduction due to the insulator layer limited, according to a method published in Appl. Phys. Lett. 110, 153504 (2017).

$${t}_{tr} = \frac{4}{π} {t}_{2 j 0} \sqrt{\frac{1}{1 + {ε}_{s} {d}_{i} / {ε}_{i} {d}_{s}}} \text{?} \qquad ( 3 )$$

$$\text{?} \text{indicates text missing or illegible when filed}$$

FIG. 6 is a graph illustrating waveforms of transmission currents observed in a case where the reverse voltage application speed A=150 kV/s and the forward voltage V_{FB} was changed from 0V to −10V, with an element having NPB (naphthyl-phenyl-biphenyl-diamine, 290 nm in film thickness) stacked as the hole transport layer 5 on the SiO₂ layer 21b. As shown in FIG. 6, with the forward voltage V_{FB}=0 V, the transmission current is constituted of a rectangular wave including only the displacement current j₀, which derives from the total capacitance of the SiO₂ layer 21b and the hole transport layer 5. This flat response indicates that no accumulated holes h⁺ exist in the hole transport layer 5.

When V_{FB}=2 to −10 V is applied, holes h⁺ are injected from the Al electrode 23, via the hole injection layer 22, into the hole transport layer 5, so as to be accumulated at the interface between the SiO₂ layer 21b and the hole transport layer 5, and thereby the current peak Δj due to the hole extraction is generated. When the forward voltage V_{FB} was increased, the current peak Δj increased, and was further saturated at jₛₐₜ=172 A/m². The transient current in the MIS-CELIV is finally limited by the displacement current of the insulator layer (the SiO₂ layer 21b).

That is, in a region of a low V_{FB} before sufficient holes h⁺ are supplied from the interface between the SiO₂ layer 21b and the hole transport layer 5, Δj linearly increases with respect to V_{FB} and eventually reaches a saturated state where Δj does not change even if V_{FB} is increased. From this, it is clear that when V_{FB}=−10 V, sufficient holes h⁺ have been accumulated at the interface between the SiO₂ layer 21b and the hole transport layer 5.

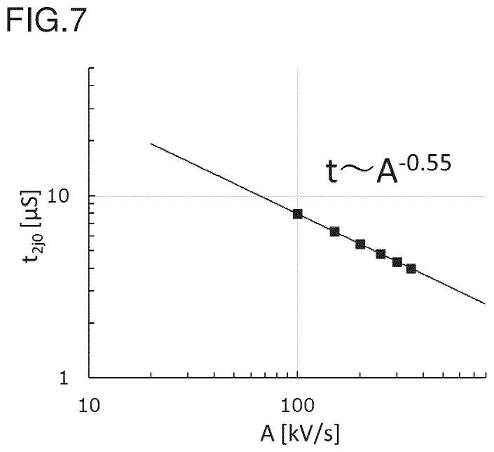

FIG. 7 is a graph illustrating electric field dependence of hole mobility estimated from waveforms of transient currents observed under conditions where the reverse voltage application speed A=100 to 350 kV/s, and the forward voltage V_{FB}=−10V. By using a formula (4) below to analyze the electric field dependence of hole mobility shown in FIG. 7, hole mobility μ₀ at electric field 0 can be calculated.

$$μ = {μ}_{0} exp ( γ \sqrt{E} ) \qquad ( 4 )$$

It should be understood that the embodiment described above is in no way meant to limit the present disclosure, which thus allows for many modifications and variations within the spirit of the present disclosure. For example, although, in the above embodiment, an example has been described where the hole transport material of the present disclosure is used as the hole transport layer 5 of the solar cell 100, the hole transport material can be used, without limitation to the solar cell 100, as a hole transport layer of, for example, a photoelectric conversion element.

An example of such a photoelectric conversion element can be produced in the following manner. For example, a semiconductor electrode (a first electrode) is formed in which a hole blocking layer and an electron transport layer that is porous are stacked on a glass substrate and further a photosensitizing material is supported. On this semiconductor electrode (the first electrode), a solution made by dissolving the hole transport material of the present disclosure in an organic solvent is applied by the spin coat method to form a hole transport layer, and on the hole transport layer formed, silver is deposited by a vacuum deposition method to form a second electrode. Below, by way of examples, the effects of the present disclosure will be described more specifically.

### Reference Example 1

### Example of Synthesis of 1,2,4,5-Tetrakis (Bromomethyl) Benzene

After placing a stirrer in a 500 mL three necked flask, attaching a reflux condenser to the flask, and purging the interior of the system with nitrogen, 13.42 g (0.1 mol) of 1,2,4,5-tetramethylbenzene was dissolved in 200 mL of ethyl acetate. 80.1 g (0.45 mol) of N-bromosuccinimide and 0.82 g (0.005 mol) of azobisisobutyronitrile were added and stirred at 70° C. for three hours. After reaction, precipitate was filtered out by filtration, and filtrate was collected and concentrated under reduced pressure in an evaporator. A crude product obtained was washed with methanol (200 mL), and then recrystallized with toluene (80 mL), as a result of which 15.3 g (0.034 mol) of 1,2,4,5-tetrakis (bromomethyl) benzene was obtained (a white solid, a yield of 34%).

### Reference Example 2

### Example of Synthesis of 1,2,4,5-Tetrakis (Diethylphosphonomethyl) Benzene

After placing a stirrer in a 300 mL three-necked flask, attaching a reflux condenser to the flask, and purging the interior of the system with nitrogen, 13.5 g (0.03 mol) of 1,2,4,5-tetrakis (bromomethyl) benzene and 29.9 g (0.18 mol) of triethyl phosphite were put in the flask and stirred at 130° C. for three hours. The reaction mixture was concentrated in an evaporator under reduced pressure, and the triethyl phosphite was removed at 95° C., as a result of which a colorless oil was obtained as a crude product. After cooling to room temperature, 50 mL of isohexane was added and stirred, as a result of which a white solid was generated. The solid generated was filtered out by filtration and washed with isohexane, as a result of which 19.0 g (0.028 mol) of 1,2,4,5-tetrakis (diethylphosphonomethyl) benzene was obtained (a white solid, a yield of 93%).

### Reference Example 3

### Example of Synthesis of 4-((4-n-Butylphenyl) (4-Methoxyphenyl)Amino)Benzaldehyde

After placing a stirrer in a 500 mL three-necked flask and purging the interior of the system with nitrogen, 6.63 g (0.020 mol) of 4-n-butyl-N-(4-methoxyphenyl)-N-phenylaniline was dissolved in 150 mL of dimethylformamide, and the solution was cooled to −10° C. Subsequently, 6.13 g (0.04 mol) of phosphoryl chloride was added to the solution and stirred at 80° C. for 24 hours. The reaction mixture was poured into 400 mL of water, and a 20% aqueous solution of sodium hydroxide was added for adjustment to pH 7 to 10. Subsequently, an organic layer was extracted twice with 50 mL of chloroform, and 10 g of anhydrous sodium sulfate was put in the organic layers and stirred at room temperature for 30 minutes. Subsequently, a precipitate was filtered out by filtration, and filtrate was collected and concentrated under reduced pressure in an evaporator. The crude product obtained was refined by silica gel column chromatography, using a developing solvent composed of a 1:1 ratio of chloroform to ethyl acetate, and was then concentrated under reduced pressure in the evaporator again at 95° C., as a result of which 6.11 g (0.017 mol) of 4-((4-n-butylphenyl) (4-methoxyphenyl) amino) benzaldehyde was obtained (a light yellow oil, a yield of 85%).

### Example 1

### Example of Synthesis of 1,2,4,5-Tetrakis (6-Diphenylamino) Styrylbenzene Derivative (HMT-1)

After placing a stirrer in a 500 mL three-necked flask, attaching a reflux condenser to the flask, and purging the interior of the system with nitrogen, 1.9 g (0.0028 mol) of 1,2,4,5-tetrakis (diethylphosphonomethyl) benzene represented by a formula (A) and 5.03 g (0.014 mol) of 4-((4-n-butylphenyl) (4-methoxyphenyl) amino) benzaldehyde represented by a formula (B) were dissolved in 200 mL of dimethylformamide, and was cooled to −10° C. Thereafter, 10.8 g (0.056 mol) of a 28% solution of sodium methylate in methanol was added, and stirred at room temperature for 12 hours.

After the reaction, the reaction mixture was put in a conical flask containing 1 L of water, and was stirred for 30 minutes. The precipitate generated was filtered outed by filtration, and washed with water and methanol. The crude product obtained was refined by silica gel column chromatography, using a developing solvent composed of a 49:1 ratio of toluene to ethyl acetate, and was concentrated under reduced pressure in an evaporator. The concentrated solution was dripped into the methanol, and a solid matter was filtered out by filtration and dried, as a result of which 1.65 g (0.0011 mol) of 1,2,4,5-tetrakis (6-diphenylamino) styrylbenzene derivative (HMT-1) was obtained (an orange-colored solid, a yield of 39%). A synthesis scheme is shown below.

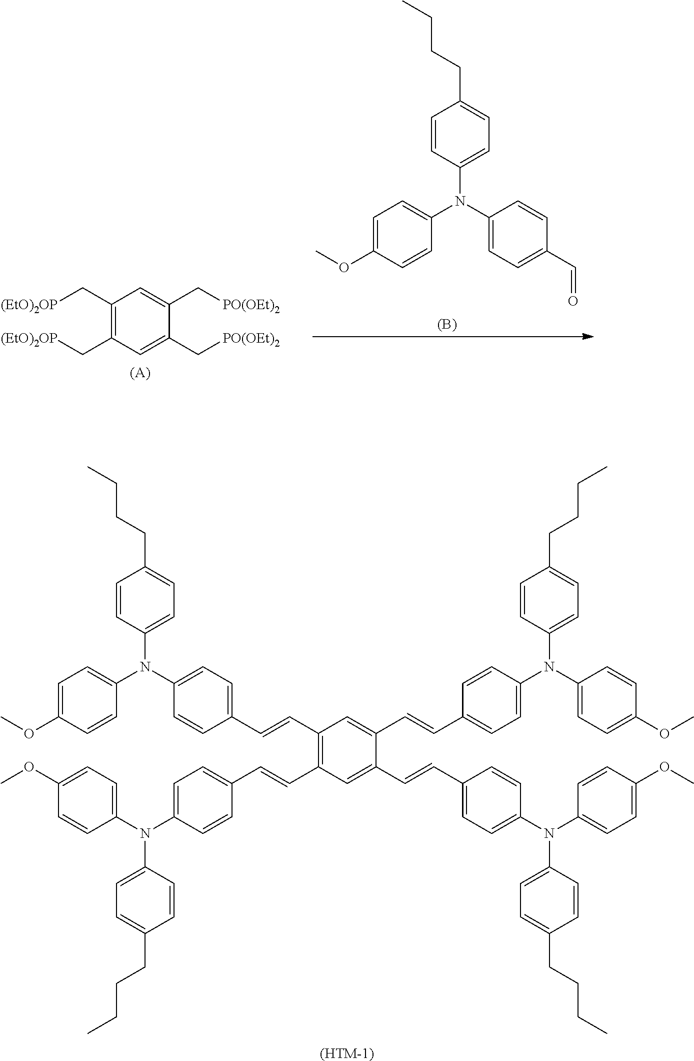

### Example 2

### Measurement of Charge Mobility of 1,2,4,5-Tetrakis (6-Diphenylamino) Styrylbenzene Derivative by the MIS-CELIV Method

Using the measurement device 20 illustrated in FIG. 3, by the MIS-CELIV, hole mobility was measured with respect to each of compounds HTM-1 to HTM-7, and Spiro OMeTAD as a comparative example.

The hole mobility was measured in the following manner. A highly n-doped silicon wafer substrate having a thermally oxidized silicon (SiO₂) layer with a film thickness of 30 nm was washed, first by ultrasonic cleaning in a 20% solution of Cica Clean in ultrapure water (20 minutes), then by ultrasonic cleaning in ultrapure water (10 minutes), then by ultrasonic cleaning in acetone (10 minutes), then by ultrasonic cleaning in IPA (isopropyl alcohol) (10 minutes), and finally by UV-ozone cleaning (20 minutes).

Onto the substrate washed, 300 μL of a 3% solution of the hole transport material in chloroform was dripped under atmospheric conditions, and a hole transport layer (approximately 200 nm in film thickness) was formed by the spin coat method, using a spin coater, at 5000 rpm, in 60 seconds.

On the hole transport layer, a hole injection layer of molybdenum trioxide (MoO₃) and an Al electrode were deposited under a vacuum of 1×10⁻³ Pa, respectively at a deposition rate of 0.1 Å/s for MoO₃ and at a deposition rate of 10 to 15 Å/s for Al, and thereby a measurement element was prepared. The element configuration was Si/SiO₂ (30 nm)/the hole transport layer (200 nm)/MoO₃ (5 nm)/Al (100 nm).

The measurement element prepared was set in the measurement device 20 illustrated in FIG. 3, t₂ⱼ₀ obtained from a transient current waveform of when a forward voltage V_{FB}=−10 V was applied under conditions of the reverse voltage application speed A=150 kV was estimated, and the hole mobility was calculated from formulae (2) and (3) described previously. The transient phenomena of the MIS-CELIV method were measured using a waveform generator (33511B, manufactured by Agilent Technologies) and an oscilloscope (DSO-X 2004A, manufactured by Agilent Technologies), under medium vacuum (<10 Pa).

Further, the compounds were compared in terms of solubility in chlorobenzene. The criteria for the solubility evaluation were set as follows: a case where a compound dissolved in chlorobenzene at a concentration of 9.09 mass % at room temperature was marked as Pass, and a case where a compound did not dissolve in chlorobenzene at a concentration of 9.09 mass % at room temperature was marked as Fail. The results are shown in Table 1 along with ionization potentials (I.P.) of the compounds.

TABLE 1

| | | | | Mobility |
|---|---|---|---|---|
| | Compound | Solubility | I.P. | (×10⁻⁴) |
| Present Disclosure | HTM-1 | Pass | 5.30 | 0.63 |
| | HTM-2 | Pass | 5.28 | 0.64 |
| | HTM-3 | Pass | 5.30 | 0.71 |
| | HTM-4 | Pass | 5.29 | 0.69 |
| | HTM-5 | Fail | 5.17 | — |
| | HTM-6 | Fail | 5.29 | — |
| | HTM-7 | Pass | 5.40 | 0.52 |
| Comparative Example | Spiro-OMeTAD | Pass | 5.13 | 0.32 |

As shown in Table 1, with the compounds HTM1 to HTM-4 and HTM-7, which are hole transport materials of the present disclosure, the hole mobilities were 0.5×10⁻⁴ or higher, and were higher than the hole mobility with Spiro-OMeTAD (0.32×10⁻⁴), which is a comparative example. Further, the compounds HTM-1 to HTM-4 and HTM-7 exhibited good solubility in chlorobenzene.

In particular, the hole mobilities exhibited with the compounds HTM-1 to HTM-4 were 0.6×10⁻⁴ or higher, which are extremely high hole mobilities. A presumable reason for this is that, as has been understood, hole mobility decreases when the R₂ group in the general formula (1) is a bulky substituent group, and thus, in each of the compounds HTM-1 to HTM-4, of each of which the R₂ group is a hydrogen atom, a methyl group, or a methoxy group, the hole mobility is higher than in the compound HTM-7, of which the R₂ group is an ethyl group.

From the above results, it has been confirmed that by using the compounds HTM-1 to HTM-4 and HTM-7 as hole transport materials, it is possible to effectively improve the energy conversion efficiency of solar cells.

It should be noted that the compounds HTM-5 and HTM-6 did not dissolve in chlorobenzene at room temperature, and thus it was impossible to measure hole mobility by the method of the present example. However, a hole transport layer can be formed of the compound HTM-5 or HTM-6 by dissolving it in chlorobenzene by heating and then applying the solution on the substrate, or, without using a solvent, by vacuum deposition.

The present disclosure is applicable to a hole transport material used as a material for hole transport layers, and a photoelectric conversion element and an organic solar cell that use the hole transport material. By using the present disclosure, it is possible to provide a hole transport material that is excellent in durability in high-temperature high-humidity environments and exhibits excellent photoelectric conversion performance even in faint light such as indoor light, and a photoelectric conversion element and an organic solar cell that use the hole transport member.

What is claimed is:

1. A hole transport material represented by a general formula (1) below: 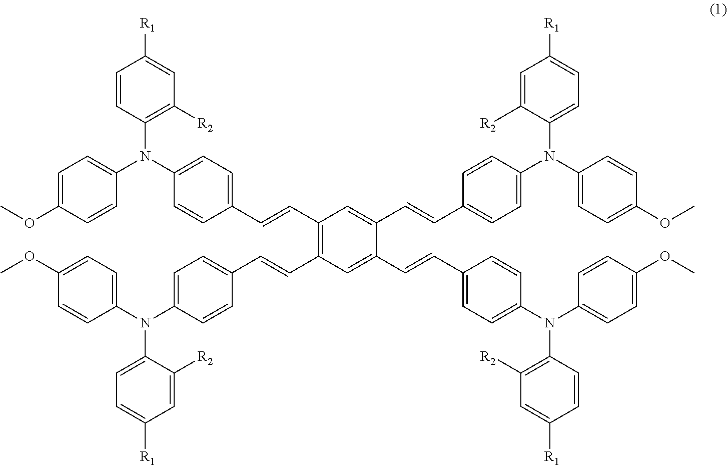
wherein
in the general formula (1), R₁ represents an alkoxy group or an alkyl group with four or fewer carbon atoms, and R₂ represents a hydrogen atom or a methyl group, and a total number of carbon atoms in R₁ and R₂ is two or more.

2. The hole transport material according to claim 1,
wherein
the general formula (1) is any one of formulae (HTM-1), (HTM-2), (HTM-3), (HTM-4), (HTM-5), (HTM-6), and (HTM-7) below.

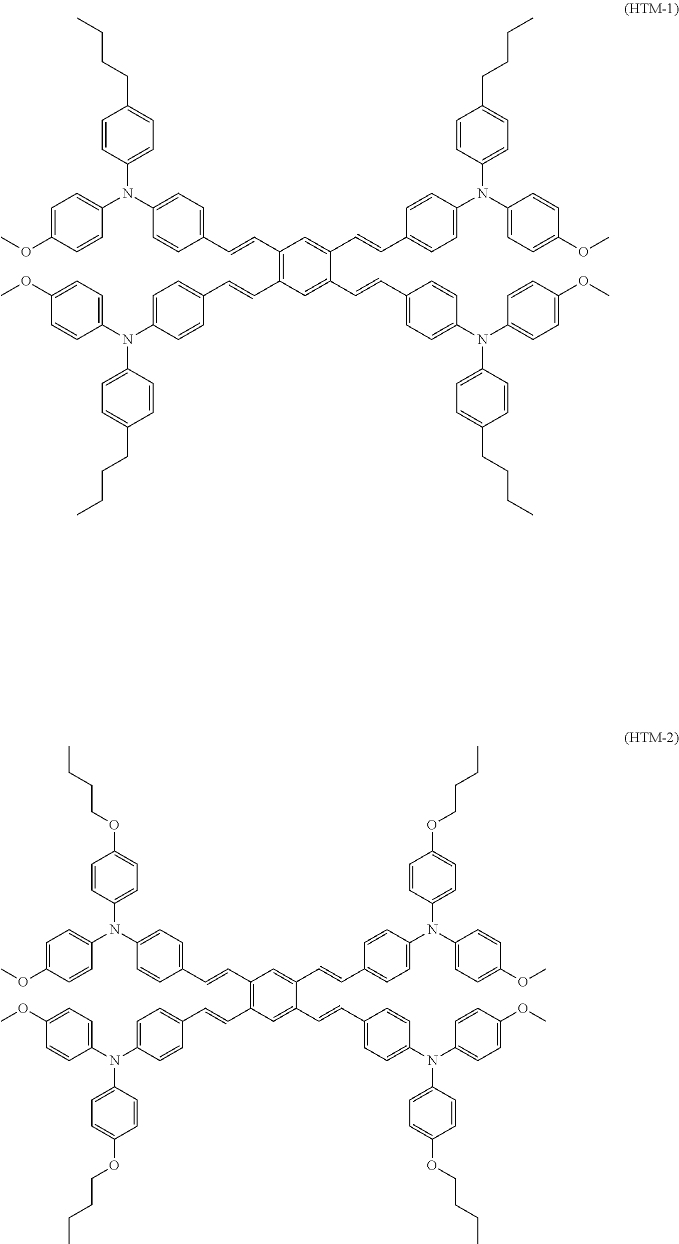

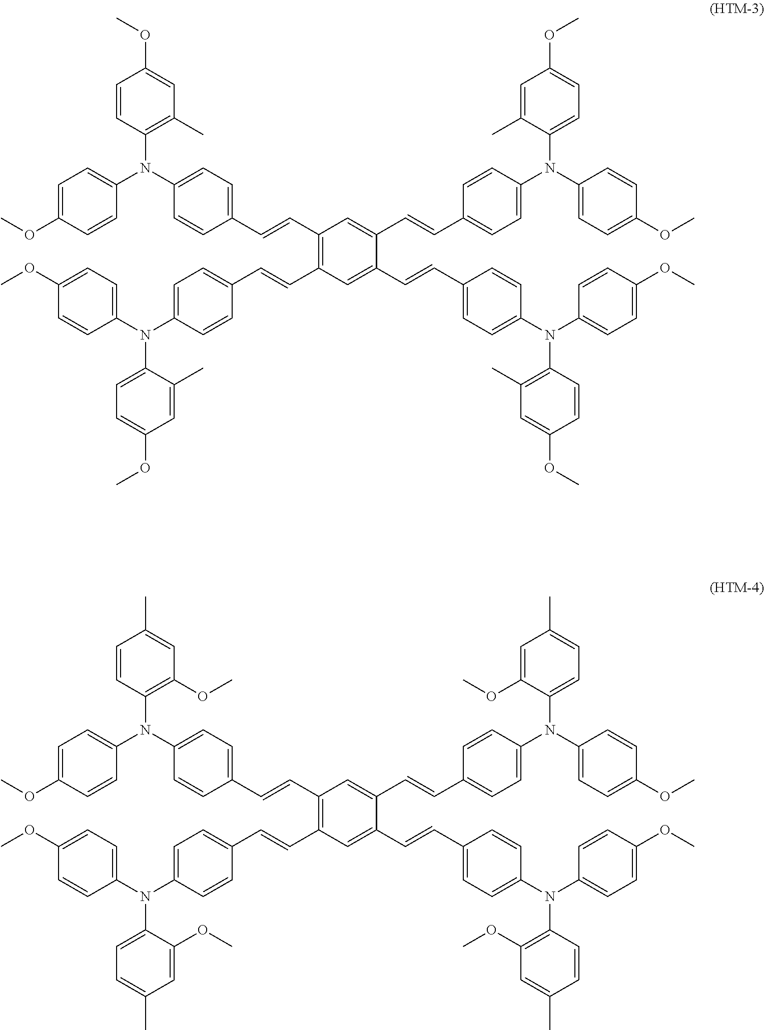

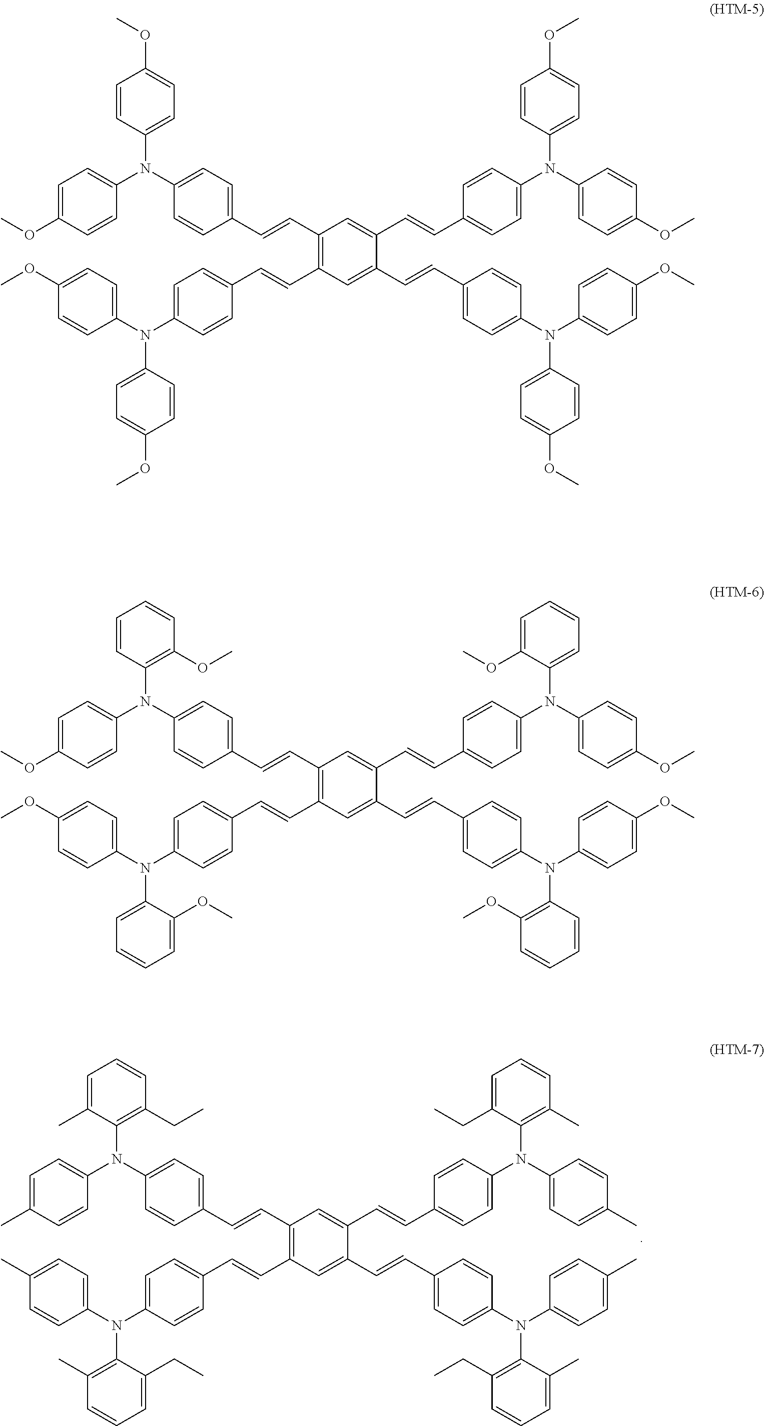

3. The hole transport material according to claim 2,
wherein
the general formula (1) is any one of the formulae (HTM-1), (HTM-2), (HTM-3), (HTM-4), and (HTM-7).

4. An organic solar cell, comprising:
a substrate;
a first electrode that is an electron injection electrode stacked on the substrate;
an electron transport layer stacked on the first electrode;
a photoelectric conversion layer stacked on the electron transport layer;
a hole transport layer stacked on the photoelectric conversion layer; and
a second electrode that is a hole injection electrode stacked on the hole transport layer,
wherein
the hole transport layer contains the hole transport material according to claim 1.

5. An organic solar cell, comprising:
a substrate;
a first electrode that is a hole injection electrode stacked on the substrate;
a hole transport layer stacked on the first electrode;
a photoelectric conversion layer stacked on the hole transport layer;
an electron transport layer stacked on the photoelectric conversion layer; and
a second electrode that is a hole injection electrode stacked on the electron transport layer,
wherein
the hole transport layer contains the hole transport material according to claim 1.

6. The organic solar cell according to claim 4,
wherein
the hole transport layer contains a hole transport material represented by any one of the formulae (HTM-1), (HTM-2), (HTM-3), (HTM-4) and (HTM-7).

7. A photoelectric conversion element, comprising:
a substrate;
a first electrode in which a hole blocking layer and an electron transport layer that is porous are stacked on the substrate, and in which further a photosensitizing material is supported;
a hole transport layer that is stacked on the first electrode; and
a second electrode that is stacked on the hole transport layer,
wherein
the hole transport layer contains the hole transport material according to claim 1.
